(19) United States
(12) Patent Application Publication
(10) Pub. No.: US 2021/0322743 A1
(43) Pub. Date: Oct. 21, 2021

(54) MICRONEEDLE ARRAY PATCH FOR DRUG DELIVERY AND PRODUCTION METHOD THEREOF

(71) Applicant: Indian Institute of Technology Bombay, Mumbai (IN)

(72) Inventors: Banerjee RINTI, Mumbai (IN); Khire Achyut SHARAD, Mumbai (IN); Osmani Riyaz Ali Mahafez ALI, Mumbai (IN); Rajput Amarjitsing PREMSINH, Mumbai (IN); Jaiswal Sanket V., Mumbai (IN)

(21) Appl. No.: 17/060,352

(22) Filed: Oct. 1, 2020

(30) Foreign Application Priority Data
Apr. 20, 2020 (IN) 202021016878

(51) Int. Cl. A61M 37/00; A61K 9/70; A61K 31/565; A61K 31/567; A61K 31/57; A61K 31/4025

(52) U.S. Cl. CPC A61M 37/0015; A61K 9/7084; A61K 31/565; A61M 2037/0053; A61K 31/57; A61K 31/4025; A61M 2037/0023; A61K 31/567

(57) ABSTRACT

The present invention provides microneedles and microneedle array patch (100) for drug delivery and production method thereof. In particular, the present invention provides a microneedle array patch (100) for drug delivery comprising: (a) a backing layer (103); (b) a plurality of dissolvable polymeric microneedles (102) fixed to said backing layer (103), said polymeric microneedles (102) being made up of polyvinyl alcohol (PVA) and gelatin, said polymeric microneedles (102) being fixed to and extending from an adhesive surface provided on the backing layer (103); and (c) drug loaded nanocarriers (101) entrapped within the matrix of said polymeric microneedles (102) for delivery by said polymeric microneedles (102) into skin; wherein said drug loaded nanocarriers (101) are selected from the group consisting of lipid nanocarriers or polymeric nanocarriers. The present invention also provides a method of producing the afore-mentioned microneedle array patch (100) for drug delivery.

### FIELD OF THE INVENTION

The present invention relates to the field of transdermal drug delivery (TDD). In particular, the present invention relates to microneedles and microneedle array patch for drug delivery and production method thereof. The present invention relates to drug loaded nanocarrier based polymeric microneedle array patch for delivery of drug such as but not limited to contraceptive agent. The present invention also relates to a method of producing the afore-mentioned microneedle array patch.

### BACKGROUND OF THE INVENTION

The remarkable physical barrier function of the skin poses a significant challenge to transdermal drug delivery. To address this challenge, a variety of microneedle-array based drug delivery devices have been developed. For example, one conventional method employs solid or hollow microneedles arrays with no active component. Such microneedle arrays can pre-condition the skin by piercing the Stratum corneum and the upper layer of epidermis to enhance percutaneous drug penetration prior to topical application of a biologic-carrier or a traditional patch. This method has been shown to significantly increase the skin's permeability; however, this method provides only limited ability to control the dosage and quantity of delivered drugs.

Another conventional method uses solid microneedles that are surface-coated with a drug. Although this method provides somewhat better dosage control, it greatly limits the quantity of drug delivered. This shortcoming has limited the widespread application of this approach and precludes, for example, the simultaneous delivery of optimal quantities of combinations of antigens and/or adjuvant in vaccine applications.

Accordingly, although transdermal delivery of drugs using microneedle-array based devices offers attractive theoretical advantages over prevailing oral and needle-based drug delivery methods, considerable practical limitations exist in the design, fabrication, and testing associated with microneedle arrays constructed using conventional processes.

The earlier reported EP patent application (EP No. EP3275431A1) studied the transdermal delivery of the lipophilic drug into the hydrophilic, hydrophobic or amphiphilic polymer which is biodegradable in the nature into the microstructure device. The microstructure device of claim wherein the microstructures are microneedles, microblades, microknives, microfibers, microspikes, microprobes, microbarbs, microarrays, or microelectrodes. The microstructures consist of Polyvinyl alcohol (PVA), Polyvinyl pyrrolidone (PVP), Hyaluronic acid (HA) and Chitosan. The microstructures are cylindrical in shape with dimensions-Height—600 μm, Tip diameter—35 μm and 5×5 in size. However, it does not teach drug loaded degradable nanoparticles/nanocarriers encapsulated within the matrix of microneedle platforms; which are conical in shape.

The patent EP2769749 is based on the manufacturing a microneedle array in which a drug is deposited on tip of the microneedles. The cone shaped microneedle consists of PTH, interferon, insulin, exendin-4, exendin derivative, EGF, FGF, botulinum toxin, various antigen proteins or virus fragments as an active pharmaceutical ingredient, hyaluronic acid, dextrin, dextran, carboxymethyl cellulose, chondroitin sulfate, proteoglycan, polyacrylic acid (salt), polyvinyl pyrrolidone, and polyvinyl alcohol as base material. The dimensions of the microneedles are height 300-500 m, base diameter: 0.16 mm, top diameter: 0.03 mm and depth: 0.8 mm, interval: 0.6 mm, rate of 250 portions per 1 cm². Microneedles are administered through transdermal route of administration. It does not teach nanoparticles/nanocarriers in microneedle platforms which are conical in shape where the drug entrapment is within degradable nanoparticles/nanocarriers throughout the matrix of the microneedles.

The researchers then studied dissolvable microneedles comprising one or more encapsulated cosmetic ingredients in EP Patent No. 2866607. A microneedle array which is capable of delivering cosmetic ingredients into the skin without safety risks associated with metal or plastic microneedles. The purpose of the present invention is to provide a microneedle array which can be easily inserted into skin, leave contained cosmetic agents under the surface of skin by dissolution, swell or break off of needles, and dissolve or disappear into skin. It consists of an applicator for applying cosmetic agents into human skin. The microneedles are cone-shaped or pyramid-shaped microneedles and tips which are knife-shaped to facilitate insertion into the skin. It consists of water-insoluble benefit agent which is selected from the group consisting of lipids, oils, waxes, proteins, hydrophobically surface-modified pigments, inorganic compounds, and mixtures thereof. The microneedle system consists of biodegradable polymer (hyaluronic acid) which is water-soluble, and in addition to the drug. It does not teach nanoparticles/nanocarriers in microneedle platforms which are conical in shape where the drug entrapment is within degradable nanoparticles/nanocarriers throughout the matrix of the microneedles.

The patent studied for the administration of the ascorbic acid (JP Patent No. 2005154321). The patent claims a means for effectively feeding vitamin C to melanocytes present in the epidermis base layer. The microneedle consists of ascorbic acid or its derivative included in microneedles the main component material of which consists of trehalose or a mixture of maltose and dextran. The microneedle is inserted into the skin and either dissolved or left in the skin to administer the skin with vitamin C. The microneedle is a pyramid shape or a prismatic shape, or, in the form of fine cylindrical or a conical shape or a needle or elongate in shape. It does not teach nanoparticles/nanocarriers in microneedle platforms which are conical in shape where the drug entrapment is within degradable nanoparticles/nanocarriers throughout the matrix of the microneedles.

The invention disclosed for a medical device in EP Patent No. EP1819393 is suitable for use in the delivery of active component into or through the skin. The microneedle is pyramidal in shape with height 250 microns, base 83.3 microns, area 2 cm², thickness 1.02 mm and array of 37×37. The medical device consists of flexible backing member consisting of polyethylene terephthalate, polycarbonate, and polyethylene. It also comprising of a pressure sensitive adhesive layer of the flexible backing member. An applicator of microneedles has tips which are knife-shaped to facilitate insertion into the skin and containing one water-insoluble agent. The water-insoluble agent consists of lipids, oils, waxes, proteins, hydrophobically surface modified pigments, inorganic compounds, and mixtures thereof. It does not teach nanoparticles/nanocarriers in microneedle platforms which are conical in shape where the drug entrapment is within degradable nanoparticles/nanocarriers throughout the matrix of the microneedles.

WO2013165715 discloses microneedle based transdermal drug delivery device and method of preparation of it. The microneedle based transdermal drug delivery device, said device comprising at least one hollow interior microneedle, with a base end and tip, mounted on the lower side of the top portion of a hinged clamshell case; said clamshell case comprising a top portion, a bottom portion, and a hinge by which said top portion may pivot with respect to said bottom portion, said clamshell case configured to administer drug to said skin of said user through the lower skin side of said bottom portion of said clamshell case. It teaches hollow microneedles. It does not teach nanoparticles/nanocarriers in microneedle platforms which are conical in shape where the drug entrapment is within degradable nanoparticles/nanocarriers throughout the matrix of the microneedles for tuneable controlled release of drug based on degradation kinetics.

Therefore, the present invention has been made in order to circumvent the above-described problems occurring in the prior art, and it is an object of the present invention to provide a microneedle array patch which is tuneable for sustained/controlled release and delivery of therapeutics/drugs across skin. It is an object of the present invention to provide a microneedle array patch (100) which incorporates a variety of therapeutic agent or drug loaded nanocarriers (101) like liposomes, niosomes, nanoparticles, solid lipid nanoparticles, nanostructured lipid carriers, nanosponges, nanosuspensions both organic as well as inorganic in nature, within the solid matrix of the polymeric microneedles (102), which are deposited directly as a depot within skin. The patch (100) can specifically be used to deliver therapeutic agents via skin with the aim of improving delivery of a contraceptive agent.

### SUMMARY OF THE INVENTION

In one aspect, the present invention provides a microneedle array patch (100) for drug delivery comprising:

(a) a backing layer (103);

(b) a plurality of dissolvable polymeric microneedles (102) fixed to said backing layer (103), said polymeric microneedles (102) being made up of polyvinyl alcohol (PVA) and gelatin, said polymeric microneedles (102) being fixed to and extending from an adhesive surface provided on the backing layer (103); and

(c) drug loaded nanocarriers (101) entrapped within the matrix of said polymeric microneedles (102) for delivery by said polymeric microneedles (102) into skin; wherein said drug loaded nanocarriers (101) are selected from the group consisting of lipid nanocarriers or polymeric nanocarriers.

In another aspect, the present invention provides a method of producing the microneedle array patch (100) comprising:

a) preparing a master mould which replicates the required microneedle array configuration;

b) preparing a secondary mould using soft-lithography of an elastomer wherein the secondary mould has the same surface contour as the master mould;

c) curing the elastomer and separating the secondary mould from the master mould;

d) preparing and mixing a base polymer matrix (102) with drug loaded nanocarriers (101);

e) pouring the base polymer matrix (102) along with drug loaded nanocarriers (101) onto the secondary mould and centrifuging the same to ensure homogenous solution enters to all the pores;

f) transferring the secondary mould centrifugally filled with base polymer matrix (102) solution containing drug loaded nanocarriers (101) in a sterile container and drying said solution for 8 hours to 48 hours at 20° C. to 40° C. with a relative humidity ranging between 45% to 55%;

g) transferring the microneedles on a backing layer (103); wherein one side of the backing layer has a pressure sensitive adhesive applied for attachment of the backing layer (103) with the polymeric microneedles (102) to form the microneedle array patch (100).

In yet another aspect, the present invention provides a method of delivering a drug into or through skin, comprising applying the afore-mentioned microneedle array patch (100) onto skin for predetermined time period and inserting at least one of the polymeric microneedles (102) into or through the skin to deliver the drug through the microneedles followed by peeling off the backing layer (103).

### BRIEF DESCRIPTION OF THE DRAWINGS

The foregoing summary, as well as the following detailed description of the invention will be better understood when read in conjunction with the appended drawings. For the purpose of assisting in the explanation of the invention, they are shown in the drawings embodiments which are presently preferred and considered illustrative. It should be understood, however, that the invention is not limited to the precise arrangements and representation shown therein.

FIG. 1: Schematic representation of drug loaded nanocarriers entrapped within the polymeric microneedles (backing layer coated with pressure sensitive adhesive).

FIG. 2: Rat cadaver skin penetration using nanocarrier loaded microneedles.

FIG. 3: Image of prototype patch of nanocarrier loaded microneedles.

FIG. 4: Scanning electron microscopic image of a single microneedle from the patch (nanocarrier loaded).

FIG. 5: Transmission electron microscopic image of liposomes within microneedles.

FIG. 6: Transmission electron microscopic image of polymeric nanoparticles within microneedles.

FIG. 7: Drug release profile of API loaded microneedles for 1 week (Composition—LNG 10 mg in PVA-Gelatin biopolymer matrix).

FIG. 8: Drug release profile of Liposomes loaded microneedles for 6 weeks (Composition-LNG loaded liposomes equivalent to 10 mg LNG dose in PVA-Gelatin biopolymer matrix).

FIG. 9: Drug release profile of nanocarrier loaded microneedles for 6 months (Composition—LNG loaded liposomes and PCL nanoparticles equivalent to 10 mg LNG dose in the ratio of 10:90 and 20:80 respectively in PVA-Gelatin biopolymer matrix).

FIG. 10: Drug release profile of PCL nanoparticle laded microneedles for 10 months (Composition—LNG loaded PCL nanoparticles equivalent to 10 mg LNG dose in PVA-Gelatin biopolymer matrix).

FIG. 11: Representative image of nanocarrier loaded microneedles pierced and separated from gelatin phantom.

### DETAILED DESCRIPTION OF THE INVENTION

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification are to be understood as being modified in all instances by the term “about”.

Thus, before describing the present invention in detail, it is to be understood that this invention is not limited to particularly exemplified process parameters that may of course, vary. It is also to be understood that the terminology used herein is for the purpose of describing particular embodiments of the invention only, and is not intended to limit the scope of the invention in any manner.

The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

It must be noted that, as used in this specification, the singular forms “a” “an” and “the” include plural referents unless the content clearly dictates otherwise. The terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the invention.

As used herein, the terms “comprising” “including,” “having,” “containing,” “involving,” and the like are to be understood to be open-ended, i.e., to mean including but not limited to.

In one aspect, the present invention provides a microneedle array patch (100) for drug delivery comprising:

(a) a backing layer (103);

(b) a plurality of dissolvable polymeric microneedles (102) fixed to said backing layer (103), said polymeric microneedles (102) being made up of polyvinyl alcohol (PVA) and gelatin, said polymeric microneedles (102) being fixed to and extending from an adhesive surface provided on the backing layer (103); and

(c) drug loaded nanocarriers (101) entrapped within the matrix of said polymeric microneedles (102) for delivery by said polymeric microneedles (102) into skin; wherein said drug loaded nanocarriers (101) are selected from the group consisting of lipid nanocarriers or polymeric nanocarriers.

In the present invention, the microneedle array patch (100) is fabricated for drug delivery to and through the skin, comprising of nanocarriers encapsulating a drug such as but not limited to a contraceptive agent, and the said nanocarriers being carried/entrapped in a polymeric matrix. To be more precise, two types of nanocarriers viz. lipid/liposomes based nanocarriers and polymer based nanocarriers are uniformly dispersed into PVA: gelatin polymeric matrix. The basis of opting to a combination of nanocarriers lies in the rapid attainment of effective plasma concentration of contraceptive agent and maintaining the same over an extended period of time, thereafter. Herein, liposomes containing contraceptive agent were loaded in microneedles for a burst release and rapid attainment of effective plasma concentration, while nanoparticles were incorporated for a slow, sustained release over a longer period of time. All components used in the preparation of the aforementioned nanocarriers (liposomes as well as nanoparticles) were biocompatible and biodegradable in nature. Moreover, the polymeric matrix components (PVA:Gelatin) are also biodegradable materials.

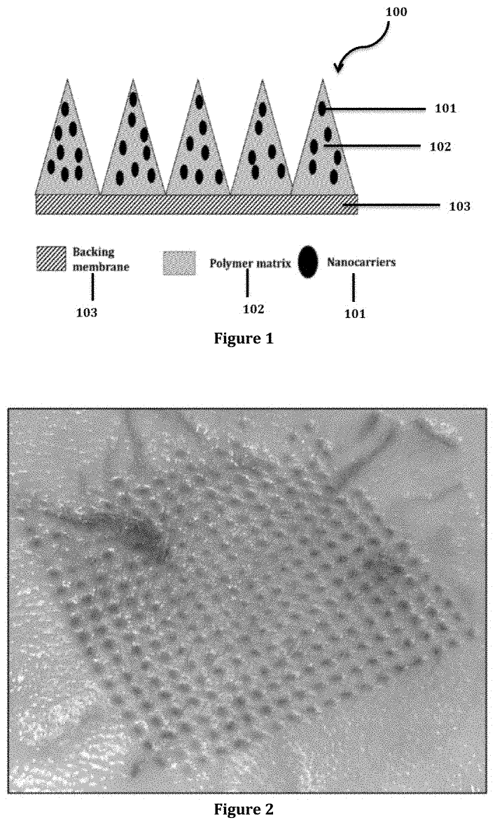

FIG. 1 is a general view or schematic representation of the microneedle array patch (100). As shown in the figure, a plurality of dissolvable polymeric microneedles (102) for skin insertion is fixed to a backing layer (103). A combination of drug loaded polymeric nanocarriers (101) and drug loaded lipid nanocarriers (101) are entrapped within the polymeric microneedle matrix (102) for delivery by said polymeric microneedles (102) into skin.

The microneedle array patch (100) of the present invention is tuneable for sustained release and delivery of drug from 1 week to 24 months.

Suitable examples of the backing layer (103) include polyester (e.g. polyethylene terephthalate; PET), cellulose acetate (0.01 mm-0.5 mm sheets), polyvinyl chloride (0.08 mm-0.5 mm sheets), fiber reinforced plastic (FRP) sheets, polypropylene; polyethylene (particularly low density, linear low density, metallocene, and high density), ethylene-vinyl acetate copolymer, polyurethane, ethyl cellulose, fabrics, non-wovens, foam, coextruded multilayer polymeric films, in an intact or perforated form.

In an embodiment of the present invention, the thickness of the backing layer (103) is in the range of 0.01 mm to 0.5 mm.

The adhesive surface comprises a pressure sensitive adhesive disposed on one side of backing layer (103) to facilitate adhesive attachment of the backing layer (103) with the polymeric microneedles (102). In an embodiment of the present invention, the pressure sensitive adhesive (PSA) is selected from the group consisting of silicones, polyacrylate adhesives (methacrylate polymers and copolymers, polyisobutylenes, polysiloxanes, polyisoprene, polybutadiene, styrenic block polymers, combinations thereof.

The polyvinyl alcohol (PVA) and gelatin are present in the ratio of 2:1.

Suitable examples of the lipid nanocarriers are selected from the group consisting of soya phosphatidylcholine (SPC), oleic acid, hydrogenated soya phosphatidylcholine (HSPC), Distearoyl phosphatidylcholine (DSPC), Dimyristoylphosphatidylcholine (DMPC), Di-oleoyl phosphatidylcholine (DOPC), 1-palmitoyl-2-oleoyl-sn-glycero-3-phosphocholine (POPC), 1,2-distearoyl-sn-glycero-3-phosphoglycerol (DSPG), 1,2-dioleoyl-sn-glycero-3-phosphoethanolamine (DOPE), egg lecithin, soya lecithin, egg phosphatidylcholine, phosphatidylethanolamine (PE), phosphatidylserine (PS), phosphatidic acid (PA), phosphatidylinositol (PI), phosphatidylglycerol (PG), cardiolipin (CL) or combinations thereof. Suitable examples of the polymeric nanocarriers are selected from the group consisting of polycaprolactone (PCL), polymethylmethacrylate (PMMA), PLA (poly-lactic acid), polyglycolic acid (PGA), polylactide-co-glycolic acid (PLGA), polyvinyl alcohol (PVA), polyvinylpyrrolidone (PVP), carboxymethyl cellulose (CMC), polyethylene (PE), polypropylene, or combinations thereof. The lipid nanocarriers and the polymeric nanocarriers of the present invention may be functionalized.

In an embodiment of the present invention, the drug is selected from the group of contraceptive agents, anti-inflammatory agents, anti-psychotic agents, anti-diabetic agents, local anesthetics, narcotics and psychotropic agents, anticoagulant medications.

Particularly, the drug is levonorgestrel, estradiol, ethinylestradiol, progestins, ormeloxifene, or combinations thereof.

In another aspect, the present invention provides a method of producing the microneedle array patch (100) comprising:

a) preparing a master mould which replicates the required microneedle array configuration;

b) preparing a secondary mould using soft-lithography of an elastomer wherein the secondary mould has the same surface contour as the master mould;

c) curing the elastomer and separating the secondary mould from the master mould;

d) preparing and mixing a base polymer matrix (102) with drug loaded nanocarriers (101);

e) pouring the base polymer matrix (102) along with drug loaded nanocarriers (101) onto the secondary mould and centrifuging the same to ensure homogenous solution enters to all the pores;

f) transferring the secondary mould centrifugally filled with base polymer matrix (102) solution containing drug loaded nanocarriers (101) in a sterile container and drying said solution for 8 hours to 48 hours at 20° C. to 40° C. with a relative humidity ranging between 45% to 55%; and

g) transferring the microneedles on a backing layer (103); wherein one side of the backing layer has a pressure sensitive adhesive applied for attachment of the backing layer (103) with the polymeric microneedles (102) to form the microneedle array patch (100).

In an embodiment of the present invention, the master mould is prepared by techniques such as EDM (electro-discharge machining), 2 photon lithography, laser writing, masked lithography. Silicone elastomer is used to prepare secondary mould.

In yet another aspect, the present invention provides a method of delivering a drug into or through skin, comprising applying the afore-mentioned microneedle array patch (100) onto skin for a predetermined time period and inserting the plurality of polymeric microneedles (102) into or through the skin to deliver the drug through the microneedles followed by peeling off the backing layer (103).

In an embodiment of the present invention, the microneedle array patch (100) may be applied on skin for about 5 to 10 minutes, and then peeled off the backing layer (103).

In an embodiment of the present invention, the polymeric microneedles (102) are peeled off or separated from the backing layer (103) after insertion into the skin due to the hydrophilic-hydrophobic interaction between the polymeric microneedles (102) and the adhesive surface of the backing layer (103). The hydrophilic-hydrophobic interaction is a result of contact angle difference between the polymeric microneedles (102) and the adhesive surface of the backing layer (103) and wherein said contact angle difference is greater than 10°.

In a preferable embodiment of the present invention, the hydrophilic-hydrophobic interaction is a result of difference in the surface energies of the material used for polymeric microneedle matrix and the pressure sensitive adhesive. The surface energy is calculated based on contact angle of water with polymer and can be calculated by Young-Dupre equation. It is widely regarded that contact angle less than 900 is considered as hydrophilic while contact angle more than 900 is hydrophobic. The polymer matrix used in the preparation of dissolvable microneedles of the present invention has a contact angle of 72-770 while for adhesive surface/layer it is 98-105°. This large difference in contact angle contributes to easy separation of microneedle layer from adhesive layer. In this present invention, adhesive layer serves two basic purposes; one to hold microneedles in stable manner and two; to get adhered onto skin allowing microneedles to be placed firmly.

Post insertion and detachment from the backing layer (103), the polymeric microneedles (102) gets dissolved gradually via surface erosion by human body intrinsic or indigenous factors.

The following examples are provided to better illustrate the claimed invention and are not to be interpreted in any way as limiting the scope of the invention. All specific materials, and methods described below, fall within the scope of the invention. These specific compositions, materials, and methods are not intended to limit the invention, but merely to illustrate specific embodiments falling within the scope of the invention. One skilled in the art may develop equivalent materials, and methods without the exercise of inventive capacity and without departing from the scope of the invention. It is the intention of the inventors that such variations are included within the scope of the invention.

### EXAMPLES

### Example 1: Preparation of Drug Loaded Nanocarrier

Drug loaded nanocarriers in form of liposomes and nanoparticles were prepared. A desired quantity of the drug, lipid and water were weighed. Then, drug and lipid were dispersed in solvent with stirring. After that, this solution was added drop wise using syringe gauge into the aqueous phase; which resulted in spherical shaped liposomes. Then, stirring was continued at 500-1000 rpm for 24 h in order to remove solvent. Polymeric nanoparticles were prepared using classic solvent injection technique. In brief, drug and polymer(s) were dissolved in an organic solvent and stabilizer(s) were dissolved in water. Controlled drop wise addition of organic phase was done to the slightly warm aqueous phase under continuous stirring at 1000-1200 rpm followed by overnight solvent evaporation.

### Example 2: Preparation of Microneedle Polymer Matrix

Microneedles have hydrophilic polymer(s)/mixture of polymer(s) matrix as base composition. The polymer matrix was made by dissolving polyvinyl alcohol and gelatin in hot water (70-75° C.). The polymers were accurately weighed and mixed in the ratio of 2:1. The prepared nanocarriers were then loaded into this polymer matrix. The mixture of polymers and nanocarrier are mixed in proportion to achieve desired drug loading concentration in microneedle, as well as to maintain desired mechanical strength of microneedle required to pierce through the skin.

### Example 3: Preparation of Master and Primary Mould

Prior to design the mould for microneedles, desired drug loading, ratio of nanocarriers to polymer matrix has been determined. This input is used to finalize dimensions of microneedle (base diameter, height, aspect ratio), array size, and pitch (distance between two microneedles).

The dimension details are as follows: Base diameter—300 micron, Height—900 micron, Aspect ratio—3:1 (Height:Base diameter), Array size—18*18, Pitch—1.5 millimeter, Patch size—35 millimeter. These parameters once finalized, the master mould which replicates the microneedle array is designed. The designed master mould has then been fabricated using suitable techniques like EDM (electro-discharge machining), 2 photon lithography, laser writing, masked lithography, and other appropriate processes. The fabricated master mould is either of aluminium, stainless steel, titanium, photo-curable resins, photoresists, silicone, and other materials depending upon manufacturing method used.

The master mould replicates the microneedle array configuration, and is then used to make a secondary mould. Secondary mould is a negative replica of master mould. Silicone elastomer is poured onto the master mould, which creates negative impressions of the master mould inside elastomer. The elastomer is then cured and separated from the master mould. The separated elastomer is a secondary mould, which shall be used to fabricate the microneedle patches.

### Example 4: Preparation of Microneedle Array Patch (Map)

Microneedle patch consists of base polymer matrix, and drug loaded nanocarriers in various proportions. The polymer matrix PVA:Gelatin is in the ratio (1:0.5 or 2:1) (weight basis ratio), Drug:Lipid (SPC) ratio is (1:5) (molecular weight basis ratio), Drug:Polymer (PCL) ratio is (1:3) (weight basis ratio). Base polymer matrix is prepared by mixing two or more biodegradable and biocompatible polymers. The selection of polymers, the ratio of their mixing, operating conditions to make uniform matrix, all were optimized to achieve necessary mechanical strength, dissolution of microneedles inside the skin, and compatibility to form uniform mixture with nanocarriers. Mechanical strength ranges from 0.1 to 1 Newton/microneedle.

Polymer matrix, homogeneously mixed with drug loaded nanocarriers (in certain ratios to achieve desired mechanical strength) poured onto the negative replica of secondary mould, and then the secondary mould is centrifuged to make sure homogenous solution enters to all the pores. After centrifugation, the mould is dried on particular temperature conditions which do not affect the properties of polymers, drugs, or nanocarriers. This step completes the formation of microneedles in the secondary mould.

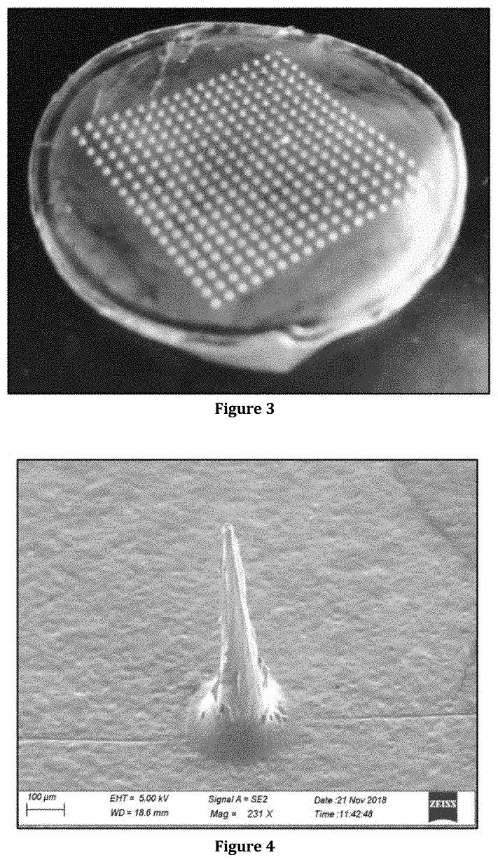

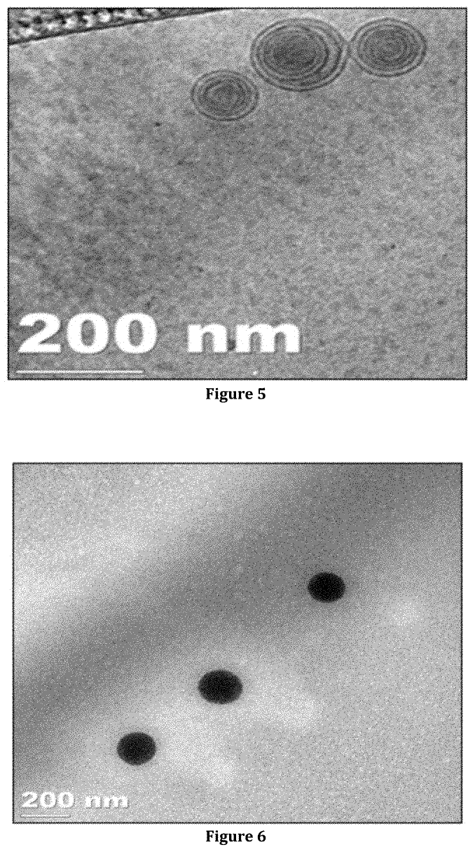

Next step is to transfer the microneedles on a backing layer. PET (Polyester) sheets of thickness around 0.3 mm was selected to act as backing layer, as it provides necessary flexibility, transparency, required for the patch. PET sheets are cut into small patch sized sheets. One side of PET is applied with pressure sensitive adhesive (PSA), and it is dried at 60° Celsius. The dried side of PET is then pressed against the face of secondary mould in which microneedles were dried. The PET sheet then pulled off from the surface of secondary mould gradually and tangentially. All the dried microneedles present in the secondary mould, are now on the PET sheet with cured PSA adhering needles to the sheet. Now, the complete microneedle patch is ready (image of a prototype is shown in FIG. 3), in which drug loaded nanocarrier are present inside the needles, and the backing layer contains all needles together with the help of PSA. In FIG. 4, SEM image of a portion of prototype microneedles patch focusing a single microneedle is shown; which depicts that the microneedles have a uniform shape and geometry. Furthermore, nanocarriers loaded microneedles are sectioned transversally using a microtome and observed under transmission electron microscope. The nanocarriers can be clearly seen in the captured TEM images (FIG. 5 and FIG. 6).

Mechanical testing of prepared microneedle patch was carried out in UTM (compressive test standard ASTM 3501), and needles were resilient to withstand forces of 0.2-1.2N/microneedle depending upon the composition of the microneedle, when subjected to compression. To determine if microneedles insert into skin, microneedles were subjected to in vitro piercing tests into cadaver rat skin. Microneedles array patch was inserted into full-thickness cadaver rat skin without subcutaneous fat that was shaved off and affixed under mild tension to a wooden plate using long screws. Microneedles were inserted by pressing against the backing layer with a thumb using a force of approximately 1.5 N and then removed immediately after the insertion. The site of microneedle insertion on the skin surface was viewed using a confocal microscope (FIG. 2).

The innovative step of the invention is separation mechanism of microneedles from backing layer. The microneedle patch shall be applied on skin for around 5 minutes, and then peel off the backing layer. During this action, all the needles should stay inside the skin, and only backing layer should come out. This is achieved by hydrophilic-hydrophobic interaction between two dissimilar polymers.

In present invention, the polymer matrix (consisting of one or more polymers) is selectively hydrophilic in nature, which helps to dissolve easily inside human skin. The pressure sensitive adhesive (PSA) applied on the backing layer is hydrophobic in nature. Hence, when a patch with microneedles made up of hydrophilic polymers, and hydrophobic backing layer is applied on the skin; the hydrophilic matrix separates from the hydrophobic base, and during peel off, only backing layer comes out.

The developed nanocarrier loaded microarray patch is non-irritant in nature when applied to the skin and having hydrophilic-hydrophobic interaction mechanism is used for the separation of the backing layer. The hydrophilic-hydrophobic interaction is a result of difference in the surface energies of the material used. The surface energy is calculated based on contact angle of water with polymer and can be calculated by Young-Dupre equation. It is widely regarded that contact angle less than 900 is considered as hydrophilic while contact angle more than 900 is hydrophobic. The polymer matrix used in the preparation of dissolvable microneedles has contact angle of 72-770 while for adhesive layer it is 98-105°. This large difference in contact angle contributes to easy separation of microneedle layer from adhesive layer. In this product, adhesive layer serves two basic purposes; one to hold microneedles in stable manner and two; to get adhered onto skin allowing microneedles to be placed firmly.

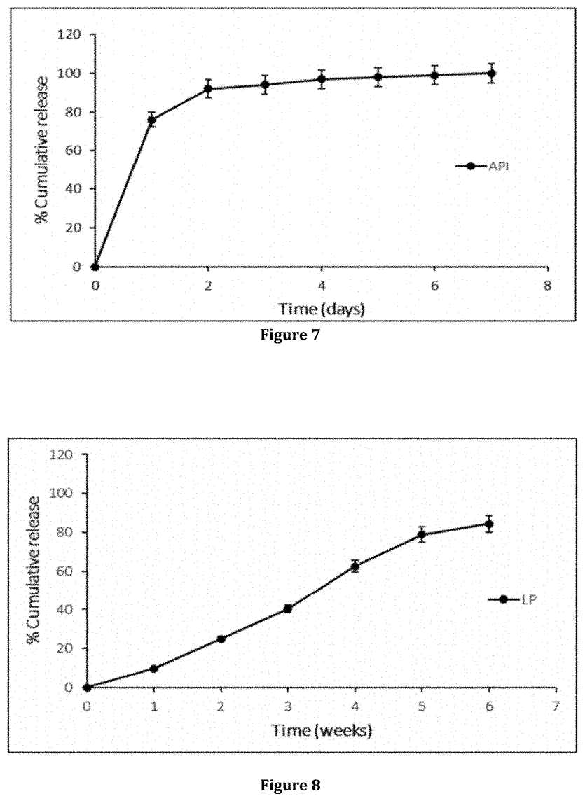

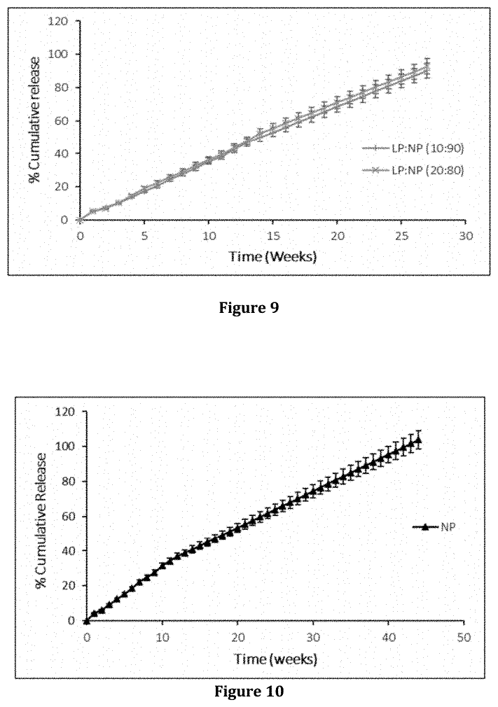

The fabricated microneedles array patches were subjected to in vitro drug release studies using modified USP Type-II dissolution apparatus. Active pharmaceutical ingredient (API) directly added to the biopolymeric microneedles have depicted release for a week (FIG. 7); which was significantly prolonged/sustained by incorporating nanocarriers, up to 6 weeks (FIG. 8), 6 months (FIG. 9) and 10 months (FIG. 10). The possible extent/degree of tunability of drug release is evident from these outcomes.

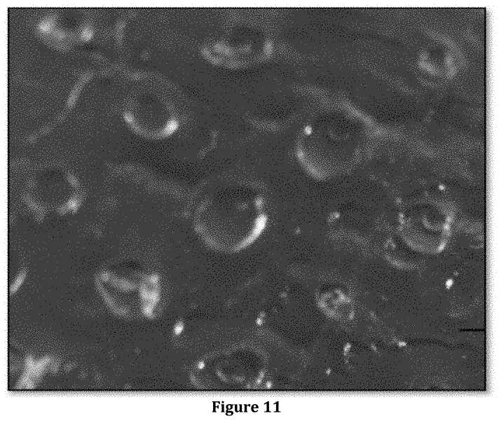

Gelatin phantom is used as a skin mimicking phantom for this study. A fabricated microneedle patch has been applied on a 5% gelatin phantom, and separation forces were applied using UTM, the backing layers gets peeled off at around 0.1-5N force depending upon the concentration of PSA on the backing layer. In the phantom, penetrated, backing layer detached microneedles were observed, whereas, peeled off layer only had traces of PSA (FIG. 11).

TABLE 1
Working formula for preparing nanocarrier loaded microneedles

| | | |
|---|---|---|
| Sr. No. | Name of Ingredient | Parts |

Nanocarriers

| | | |
|---|---|---|
| 1 | Levonorgestrel | 10 |
| 2 | Soy PC* | 35 |
| 3 | Oleic acid* | 15 |
| 4 | Polycaprolactone | 30 |
| 5 | Polyvinyl alcohol | 3 |

Microneedles Polymeric Matrix

| | | |
|---|---|---|
| 6 | Polyvinyl alcohol | 2 |
| 7 | Gelatin (Type A) | 1 |

All quantities are mentioned in dry weight basis. *Quantity measured on molecular weight basis.

Oleic acid used in the above formulation herein functions as a stabilizer, anti-oxidant agent and sorption promoter or accelerant. Oleic acid, being an unsaturated fatty acid, has a low transition temperature, and the kink in the cis-alkenyl chain of oleic acid enhances its fluidizing effects and penetration through barrier layer of skin. It also aid in reducing the liposomes rigidity which further enhances permeation to and through skin.

We claim:

1. A microneedle array patch (100) for drug delivery comprising:
(a) a backing layer (103);
(b) a plurality of dissolvable polymeric microneedles (102) fixed to said backing layer (103), said polymeric microneedles (102) being made up of polyvinyl alcohol (PVA) and gelatin, said polymeric microneedles (102) being fixed to and extending from an adhesive surface provided on the backing layer (103); and
(c) drug loaded nanocarriers (101) entrapped within the matrix of said polymeric microneedles (102) for delivery by said polymeric microneedles (102) into skin; wherein said drug loaded nanocarriers (101) are selected from the group consisting of lipid nanocarriers or polymeric nanocarriers.

2. The microneedle array patch (100) as claimed in claim 1, wherein the backing layer (103) is made up of polyester, cellulose acetate, polyvinyl chloride, fiber reinforced plastic (FRP) sheets, polypropylene; polyethylene, ethylene-vinyl acetate copolymer, polyurethane, ethyl cellulose, fabrics, non-wovens, foam, coextruded multilayer polymeric films, in an intact or perforated form.

3. The microneedle array patch (100) as claimed in claim 1, wherein the adhesive surface comprises a pressure sensitive adhesive disposed on one side of backing layer (103) to facilitate adhesive attachment of the backing layer (103) with the polymeric microneedles (102).

4. The microneedle array patch (100) as claimed in claim 3, wherein the pressure sensitive adhesive (PSA) is selected from the group consisting of silicones, polyacrylate adhesives, methacrylate polymers and copolymers, polyisobutylenes polysiloxanes, polyisoprene, polybutadiene, styrenic block polymers or combinations thereof.

5. The microneedle array patch (100) as claimed in claim 1, wherein the polymeric microneedles (102) are separated from the backing layer (103) after insertion into the skin due to the hydrophilic-hydrophobic interaction between the polymeric microneedles (102) and the adhesive surface of the backing layer (103).

6. The microneedle array patch (100) as claimed in claim 5, wherein the hydrophilic-hydrophobic interaction is a result of contact angle difference between the polymeric microneedles (102) and the adhesive surface of the backing layer (103) and wherein said contact angle difference is greater than 10°.

7. The microneedle array patch (100) as claimed in claim 1, wherein the polyvinyl alcohol (PVA) and gelatin are present in the ratio of 2:1.

8. The microneedle array patch (100) as claimed in claim 1, wherein the lipid nanocarriers are selected from the group consisting of soya phosphatidylcholine (SPC), oleic acid, hydrogenated soya phosphatidylcholine (HSPC), Di-stearoyl phosphatidylcholine (DSPC), Dimyristoylphosphatidylcholine (DMPC), Di-oleoyl phosphatidylcholine (DOPC), 1-palmitoyl-2-oleoyl-sn-glycero-3-phosphocholine (POPC), 1,2-distearoyl-sn-glycero-3-phosphoglycerol (DSPG), 1,2-dioleoyl-sn-glycero-3-phosphoethanolamine (DOPE), egg lecithin, soya lecithin, egg phosphatidylcholine, phosphatidylethanolamine (PE), phosphatidylserine (PS), phosphatidic acid (PA), phosphatidylinositol (PI), phosphatidylglycerol (PG), cardiolipin (CL) or combinations thereof.

9. The microneedle array patch (100) as claimed in claim 1, wherein the polymeric nanocarriers are selected from the group consisting of polycaprolactone (PCL), polymethylmethacrylate (PMMA), poly lactic acid (PLA), polyglycolic acid (PGA), polylactide-co-glycolic acid (PLGA), polyvinyl alcohol (PVA), polyvinylpyrrolidone (PVP), carboxymethyl cellulose (CMC), polyethylene (PE), polypropylene, or combinations thereof.

10. The microneedle array patch (100) as claimed in claim 1, wherein the drug is selected from the group of contraceptive agents, anti-inflammatory agents, anti-psychotic agents, anti-diabetic agents, local anesthetics, narcotics, psychotropic agents and anticoagulant medications.

11. The microneedle array patch (100) as claimed in claim 10, wherein the drug is levonorgestrel, estradiol, ethinylestradiol, progestins, ormeloxifene, or combinations thereof.

12. The microneedle array patch (100) as claimed in claim 1, wherein the patch (100) is tuneable for sustained release and delivery of drug from 1 week to 24 months.

13. A method of producing the microneedle array patch (100) comprising:
a) preparing a master mould which replicates the required microneedle array configuration;
b) preparing a secondary mould using soft-lithography of an elastomer wherein the secondary mould has the same surface contour as the master mould;
c) curing the elastomer and separating the secondary mould from the master mould;
d) preparing and mixing a base polymer matrix (102) with drug loaded nanocarriers (101);
e) pouring the base polymer matrix (102) along with drug loaded nanocarriers (101) onto the secondary mould and centrifuging the same to ensure homogenous solution enters to all the pores;
f) transferring the secondary mould centrifugally filled with base polymer matrix (102) solution containing drug loaded nanocarriers (101) in a sterile container and drying said solution for 8 hours to 48 hours at 20° C. to 40° C. with a relative humidity ranging between 45% to 55%;
g) transferring the microneedles on a backing layer (103); wherein one side of the backing layer has a pressure sensitive adhesive applied for attachment of the backing layer (103) with the polymeric microneedles (102) to form the microneedle array patch (100).

14. The method as claimed in claim 13, wherein the master mould is prepared by techniques such as EDM (electro-discharge machining), 2 photon lithography, laser writing, masked lithography.

15. The method as claimed in claim 13, wherein silicone elastomer is used to prepare secondary mould.
